(19) United States
(12) Patent Application Publication
(10) Pub. No.: US 2022/0133857 A1
(43) Pub. Date: May 5, 2022

(54) ORAL FORMULATION AND TREATMENT OF PTH ANALOG

(71) Applicant: ANYA BIOPHARM INC., Taipei (TW)

(72) Inventors: Siegfried GSCHLIESSER, Taipei City (TW); Bhushan Dhruvkumar DESAI, Maharashtra (TW)

(73) Assignee: ANYA BIOPHARM INC., Taipei (TW)

(21) Appl. No.: 17/436,416

(22) PCT Filed: Mar. 6, 2020
(86) PCT No.: PCT/IB2020/051958

§ 371 Date: Sep. 3, 2021

(30) Foreign Application Priority Data
Mar. 8, 2019 (IN) 201921009082

(51) Int. Cl. A61K 38/29; A61K 33/24; A61K 9/48

(52) U.S. Cl. CPC A61K 38/29; A61K 9/4816; A61K 33/24

(57) ABSTRACT

The present invention relates to method of preventing or treating disease or disorder comprising administration of oral pharmaceutical composition comprising therapeutically effective amount of PTH analog and at least one degradation preventing agent, wherein said composition provides relative bioavailability of at least 0.5%, compare to subcutaneous administration. The present invention also relates to the oral pharmaceutical composition comprising therapeutically effective amount of PTH analog and least one degradation preventing agent in physically separated dosage form and wherein said PTH analogue is in enteric coated form. At least one degradation preventing agent includes combination of at least one metal containing compound and at least one reducing agent where in at least one metal containing compound is selected for group consisting of vanadium containing compound, chromium containing compound and manganese containing compound. Preferably PTH analogue are teriparatide or abaloparatide.

### FIELD OF THE INVENTION

The present invention relates to method of preventing or treating disease or disorder comprising administration of oral pharmaceutical composition comprising therapeutically effective amount of PTH analog wherein said composition provides relative bioavailability of at least 0.5%, compare to subcutaneous administration. The present invention also relates to method of preventing or treating disease or disorder comprising administration of oral pharmaceutical composition comprising therapeutically effective amount PTH analog and at least one degradation preventing agent. The present invention also relates to the oral pharmaceutical composition comprising therapeutically effective amount of PTH analog and least one degradation preventing agent in physically separated dosage form. At least one degradation preventing agent includes combination of at least one metal containing compound and at least one reducing agent where in at least one metal containing compound is selected for group consisting of vanadium containing compound, chromium containing compound and manganese containing compound.

### BACKGROUND OF THE INVENTION

Human parathyroid hormone (PTH) is secreted by the parathyroid glands. It is a 84 amino acids circulating hormone that acts as the regulator of calcium metabolism by directly targeting bone, kidney, and intestine.

PTH Analog/s are the active substance having at least partial structural similarity with PTH and acts at least partially similar to parathyroid hormone (PTH). PTH analogues are anabolic agents developed to enhance uptake of calcium, stimulate new bone formation and reduces risk of osteoporosis fracture. PTH analog as per present invention includes teriparatide and abaloparatide.

Teriparatide, also called PTH (1-34), is human parathyroid hormone (1-34). It has an identical sequence to the 34 N-terminal amino acids of the 84-amino acid human parathyroid hormone which is the biologically active region of PTH. Currently, it is commercially available as multi-dose prefilled delivery pen for subcutaneous administration (FORTEO by Eli Lilly). Forteo contains teriparatide prepared by recombinant DNA technology. Teriparatide is currently approved for the treatment of following indications in dosage of 20 mcg of teriparatide per dose each day:
1. Treatment of Postmenopausal Women with Osteoporosis at High Risk for Fracture.
2. Increase of Bone Mass in Men with Primary or Hypogonadal Osteoporosis at High Risk for Fracture.
3. Treatment of Men and Women with Glucocorticoid Induced Osteoporosis at High Risk for Fracture.

Abaloparatide is an analog of human parathyroid hormone related peptide, PTHrP(1-34). It has 41% homology to hPTH(1-34) (human parathyroid hormone 1-34) and 76% homology to hPTHrP(1-34) (human parathyroid hormone-related peptide 1-34). Abaloparatide is currently approved for the treatment of for the treatment of postmenopausal women with osteoporosis at high risk for fracture, in dosage of 80 mcg subcutaneously once daily. It is currently available as pre-assembled single patient use disposable pen for subcutaneous administration.

Both teriparatide and abaloparatide are available for subcutaneous daily administration. Subcutaneous administration requires daily injection which is painful and is likely to have reduced patient compliance. Oral route is a simple, convenient and most preferred route for administration of a therapeutic agent. However, degradation of peptides in gastrointestinal tract prevents their absorption as an intact entity. Thus, enzymatic degradation in the gastrointestinal tract and poor permeability through the epithelial cells are the main reasons for their low oral bioavailability.

US20070155664A1 relates to combination of an effective amount of a calcium-containing compound and an effective amount of PTH wherein PTH is administered in delayed release form. It discloses use of conventional stabilizing agent which are unsuccessful so far.

U.S. Pat. No. 8,110,547B2 relates to buccal delivery of PTH components with delivery agents like 4-MOAC and 5-CNAC.

U.S. Pat. No. 9,566,246B2 relates to pharmaceutical compositions with a therapeutically effective amount of a therapeutic agent and at least one salt of a medium chain fatty acid and a hydrophobic medium, e.g. castor oil or glyceryl tricaprylate or a mixture thereof.

US20110046059A1 relates to pharmaceutical compositions comprising a pharmaceutically effective amount of a peptidyl drug and a bioavailability enhancer.

US20170304195A1 relates to pharmaceutical compositions comprising a peptide or protein drug in combination with a pharmaceutically acceptable copper salt/complex and/or a pharmaceutically acceptable zinc salt/complex, and with a pharmaceutically acceptable reducing agent. However, copper and zinc are associated with many metabolic pathways in mammals, and hence, utilization thereof for a long-term therapy may results in negative interactions.

WO2017060500A1 related to pharmaceutical compositions comprising peptide drug in combination with a pharmaceutically acceptable copper salt/complex and/or a pharmaceutically acceptable zinc salt/complex and/or a pharmaceutically acceptable iron salt/complex and a pharmaceutically acceptable complexing agent. However, copper and zinc are associated with many metabolic pathways in mammals, and hence, utilization thereof for a long-term therapy may results in negative interactions.

WO2018033927A1 relates to a pharmaceutical composition multi-unit dosage form comprising at least two discrete unit dosage forms bound to one another by a coating and/or matrix, each of said unit dosage forms comprising a therapeutically active agent and an absorption enhancer.

WO2018043942A1 relates to a complex formed by parathyroid hormone (PTH) or the fragment teriparatide (PTH(1-34)) thereof, and a deoxycholic acid derivative.

US20060252686A1 relates to combination of chromium or vanadium with antidiabetic agents for glucose disorders.

Currently no oral formulations of PTH analog are available in market. Thus, there is a need to develop oral formulations of PTH analogs with an improved bioavailability and thereby enhanced patient compliance.

### SUMMARY OF THE INVENTION

In one embodiment, the present invention relates to a method of preventing or treating disease or disorder comprising administration of oral pharmaceutical 25 composition comprising therapeutically effective amount of PTH analog to a patient in need thereof, wherein said composition provides relative bioavailability of at least 0.5%, 0.5-10%, 1-9%, 2-8%, 3-7%, 4-6% compare to subcutaneous administration.

In one embodiment, the present invention relates to a method of preventing or treating disease or disorder comprising administration of oral pharmaceutical composition comprising therapeutically effective amount of teriparatide or abaloparatide to a patient in need thereof, wherein said composition provides relative bioavailability of at least 0.5%, 0.5-10%, 1-9%, 2-8%, 3-7%, 4-6% compare to subcutaneous administration.

In one embodiment, the present invention relates to a method of preventing or treating disease or disorder comprising administration of oral pharmaceutical composition comprising therapeutically effective amount of PTH analog to a patient in need thereof, wherein said composition provides relative bioavailability of at least 0.5%, 0.5-10%, 1-9%, 2-8%, 3-7%, 4-6% compare to subcutaneous administration and said oral pharmaceutical composition achieves maximum plasma concentration (Tmax) between 45 min and 90 min.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5%, 0.5-10%, 1-9%, 2-8%, 3-7%, 4-6% compare to subcutaneous administration and said oral pharmaceutical composition achieves maximum plasma concentration (Tmax) between 45 min and 90 min.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one degradation preventing agent.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least one degradation preventing agent.

In one embodiment, the present invention relates to a method of preventing or treating disease or disorder comprising administration of oral pharmaceutical composition comprising therapeutically effective amount of PTH analogue, to a patient in need thereof, wherein said composition provides relative bioavailability of at least 0.5% compare to subcutaneous administration and wherein said oral pharmaceutical composition further comprises at least one degradation preventing agent, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

In one embodiment, the present invention relates to a method of preventing or treating disease or disorder comprising administration of oral pharmaceutical composition comprising therapeutically effective amount of teriparatide or abaloparatide to a patient in need thereof, wherein said composition provides relative bioavailability of at least 0.5% compare to subcutaneous administration and wherein said oral pharmaceutical composition further comprises at least one degradation preventing agent, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least vanadium containing compound selected from a group consisting of vanadium (V) oxide, sodium vanadate, vanadium sulfate, vanadyl sulfate, vanadium biguanide, bis(maltolato)oxavanadium (IV), vanadium acetate, vanadyl picolinate and vanadyl citrate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least chromium containing compound is selected from a group consisting of chromium picolinate, chromium polynicotinate, chromium nicotinate, chromium chloride and chromium acetate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least manganese containing compound selected from group consisting of manganese gluconate, manganese sulfate, potassium permanganate and manganese chloride, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least vanadium containing compound selected from a group consisting of vanadium (V) oxide, sodium vanadate, vanadium sulfate, vanadyl sulfate, vanadium biguanide, bis(maltolato)oxavanadium (IV), vanadium acetate, vanadyl picolinate and vanadyl citrate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least chromium containing compound is selected from a group consisting of chromium picolinate, chromium polynicotinate, chromium nicotinate, chromium chloride and chromium acetate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least manganese containing compound selected from group consisting of manganese gluconate, manganese sulfate, potassium permanganate and manganese chloride, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least metal containing compound selected from group consisting of vanadium sulfate, chromium picolinate and manganese gluconate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least metal containing compound selected from group consisting of vanadium sulfate, chromium picolinate and manganese gluconate, in any form including salts or complex thereof, at least one reducing agent and optionally one or more selected from group consisting of absorption enhancer, Pgp efflux inhibitor.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent selected from any or a combination of ascorbic acid, reduced glutathione, cysteine, uric acid, reducing sugar, glyceraldehyde, α-tocopherol, vitamin A, α-lipoic acid, dihydro-α-lipoic acid, glucose, galactose, lactose, maltose, thiol bearing compound, a thiomer and pharmaceutically acceptable salts thereof.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent selected from any or a combination of ascorbic acid, reduced glutathione, cysteine, uric acid, reducing sugar, glyceraldehyde, α-tocopherol, vitamin A, α-lipoic acid, dihydro-α-lipoic acid, glucose, galactose, lactose, maltose, thiol bearing compound, a thiomer and pharmaceutically acceptable salts thereof.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent and one or more absorption enhancer selected from group consisting of labrasol, solutol and Vitamin E.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent and one or more absorption enhancer selected from group consisting of labrasol, solutol and Vitamin E.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, at least one reducing agent and at least one P-glycoprotein (P-gp) efflux inhibitor.

In one embodiment, the present invention relates to a method of preventing or treating disease or disorder comprising administration of oral pharmaceutical composition comprising therapeutically effective amount of teriparatide or salts or complex thereof, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, at least one reducing agent and at least one P-glycoprotein (P-gp) efflux inhibitor.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said PTH analogue and said at least one metal containing compound in form of any or a combination of a salt thereof and a complex thereof, are present in physically separated form in said pharmaceutical composition.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said teriparatide or salts or complex thereof, and said at least one metal compound in form of any or a combination of a salt thereof and a complex thereof are present in physically separated form in said pharmaceutical composition.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said PTH analogue and said at least one metal containing compound in form of any or a combination of a salt thereof and a complex thereof, are present in physically separated form in said pharmaceutical composition, and wherein said PTH analogue contained in enteric coated dosage form, and at least one metal containing compound and at least one reducing agent is in enteric coated form.

In one embodiment, the present invention relates to a method of preventing or treating disease or disorder comprising administration of oral pharmaceutical composition comprising therapeutically effective amount of teriparatide or abaloparatide, to a patient in need thereof, wherein said composition provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said teriparatide or salts or complex thereof, and said at least one metal compound in form of any or a combination of a salt thereof and a complex thereof are present in physically separated form in said pharmaceutical composition, and wherein said PTH analogue contained in enteric coated dosage form, and at least one metal containing compound and at least one reducing agent is in enteric coated form.

In one embodiment, the present invention relates to an oral pharmaceutical composition comprising therapeutically effective amount of PTH analog, wherein said composition upon oral administration provides relative bioavailability of at least 0.5%, 0.5-10%, 1-9%, 2-8%, 3-7%, 4-6% compare to subcutaneous administration.

In one embodiment, the present invention relates to an oral pharmaceutical composition comprising therapeutically effective amount of teriparatide or abaloparatide, wherein said composition upon oral administration provides relative bioavailability of at least 0.5%, 0.5-10%, 1-9%, 2-8%, 3-7%, 4-6% compare to subcutaneous administration.

In one embodiment, the present invention relates to an oral pharmaceutical composition comprising therapeutically effective amount of PTH analog, wherein said composition upon oral administration provides relative bioavailability of at least 0.5%, 0.5-10%, 1-9%, 2-8%, 3-7%, 4-6% compare to subcutaneous administration and said oral pharmaceutical composition provides maximum plasma concentration (Tmax) between 45 min and 90 min.

In one embodiment, the present invention relates to an oral pharmaceutical composition comprising therapeutically effective amount of teriparatide or abaloparatide, wherein said composition upon oral administration provides relative bioavailability of at least 0.5%, 0.5-10%, 1-9%, 2-8%, 3-7%, 4-6% compare to subcutaneous administration and said oral pharmaceutical composition provides maximum plasma concentration (Tmax) between 45 min and 90 min.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one degradation preventing agent.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one degradation preventing agent.

In one embodiment, the present invention relates to an oral pharmaceutical composition comprising therapeutically effective amount of PTH analogue, wherein said composition upon oral administration provides relative bioavailability of at least 0.5% compare to subcutaneous administration and wherein said oral pharmaceutical composition further comprises at least one degradation preventing agent, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one degradation preventing agent, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least vanadium containing compound selected from a group consisting of vanadium (V) oxide, sodium vanadate, vanadium sulfate, vanadyl sulfate, vanadium biguanide, bis(maltolato)oxavanadium (IV), vanadium acetate, vanadyl picolinate and vanadyl citrate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least chromium containing compound is selected from a group consisting of chromium picolinate, chromium polynicotinate, chromium nicotinate, chromium chloride and chromium acetate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least manganese containing compound selected from group consisting of manganese gluconate, manganese sulfate, potassium permanganate and manganese chloride, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of teriparatide or abaloparatide, wherein said composition provides relative bioavailability of at least 0.5% compare to subcutaneous administration and wherein said oral pharmaceutical composition further comprises at least vanadium containing compound selected from a group consisting of vanadium (V) oxide, sodium vanadate, vanadium sulfate, vanadyl sulfate, vanadium biguanide, bis(maltolato)oxavanadium (IV), vanadium acetate, vanadyl picolinate and vanadyl citrate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least chromium containing compound is selected from a group consisting of chromium picolinate, chromium polynicotinate, chromium nicotinate, chromium chloride and chromium acetate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least manganese containing compound selected from group consisting of manganese gluconate, manganese sulfate, potassium permanganate and manganese chloride, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least metal containing compound selected from group consisting of vanadium sulfate, chromium picolinate and manganese gluconate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least metal containing compound selected from group consisting of vanadium sulfate, chromium picolinate and manganese gluconate, in any form including salts or complex thereof, at least one reducing agent and optionally one or more selected from group consisting of absorption enhancer, Pgp efflux inhibitor.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent selected from any or a combination of ascorbic acid, reduced glutathione, cysteine, uric acid, reducing sugar, glyceraldehyde, α-tocopherol, vitamin A, α-lipoic acid, dihydro-α-lipoic acid, glucose, galactose, lactose, maltose, thiol bearing compound, a thiomer and pharmaceutically acceptable salts thereof.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent selected from any or a combination of ascorbic acid, reduced glutathione, cysteine, uric acid, reducing sugar, glyceraldehyde, α-tocopherol, vitamin A, α-lipoic acid, dihydro-α-lipoic acid, glucose, galactose, lactose, maltose, thiol bearing compound, a thiomer and pharmaceutically acceptable salts thereof.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent and one or more absorption enhancer selected from group consisting of labrasol, solutol and Vitamin E.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, and at least one reducing agent and one or more absorption enhancer selected from group consisting of labrasol, solutol and Vitamin E.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, at least one reducing agent and at least one P-glycoprotein (P-gp) efflux inhibitor.

In one embodiment, the present invention relates to an oral pharmaceutical composition comprising therapeutically effective amount of teriparatide or salts or complex thereof, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof, at least one reducing agent and at least one P-glycoprotein (P-gp) efflux inhibitor

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said PTH analogue and said at least one metal containing compound in form of any or a combination of a salt thereof and a complex thereof, are present in physically separated form in said pharmaceutical composition.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said teriparatide or salts or complex thereof, and said at least one metal compound in form of any or a combination of a salt thereof and a complex thereof are present in physically separated form in said pharmaceutical composition.

In one embodiment, the present invention relates to an oral pharmaceutical composition comprising therapeutically effective amount of PTH analog, wherein said composition provides upon oral administration relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said PTH analogue and said at least one metal containing compound in form of any or a combination of a salt thereof and a complex thereof, are present in physically separated form in said pharmaceutical composition, and wherein said PTH analogue contained in enteric coated dosage form, and at least one metal containing compound and at least one reducing agent is in enteric coated form.

In one embodiment, the present invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one metal containing compound selected from group consisting of vanadium containing compound, chromium containing compound and manganese containing compound, wherein said metal containing compound is in any form including salts or complex thereof and at least one reducing agent, wherein said teriparatide or salts or complex thereof, and said at least one metal compound in form of any or a combination of a salt thereof and a complex thereof are present in physically separated form in said pharmaceutical composition, and wherein said PTH analogue contained in enteric coated dosage form, and at least one metal containing compound and at least one reducing agent is in enteric coated form.

In one embodiment, the present invention provides an oral pharmaceutical composition comprising teriparatide, wherein the composition exhibits an Cmax of about 2 ng/ml to about 10 ng/ml of teriparatide following administration of the composition to a subject.

In one embodiment, the present invention provides an oral pharmaceutical composition comprising PTH analog, Ascorbate Sodium and Vanadium Sulphate.

In one embodiment, the present invention provides an oral pharmaceutical composition comprising PTH analog, Ascorbate Sodium and Vanadium Oxide.

In one embodiment, the present invention provides an oral pharmaceutical composition comprising PTH analog, Uric Acid and Sodium Vanadate.

In one embodiment, the present invention provides an oral pharmaceutical composition comprising PTH analog, Ascorbate Sodium and Manganese Gluconate.

In one embodiment, the present invention provides an oral pharmaceutical composition comprising PTH analog, Reduced Glutathione and Chromium Picolinate.

### BRIEF DESCRIPTION OF THE DRAWINGS

FIG. 1 illustrates a graph depicting conc. vs. time profile of teriparatide (ng/ml) from different formulations, in accordance with an embodiment of the present disclosure.

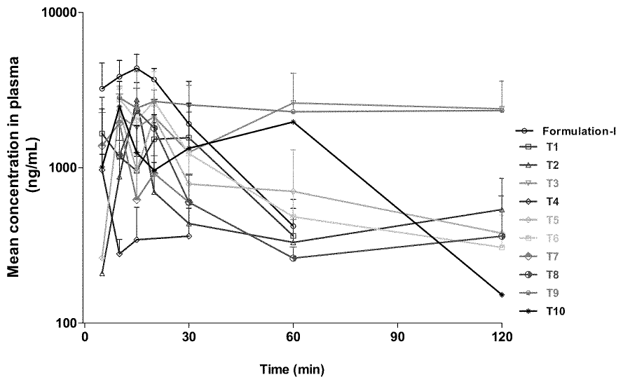

### DETAILED DESCRIPTION OF THE INVENTION

The term “PTH Analog” mean an active substance having at least partial structural similarity with PTH and acts at least partially similar to parathyroid hormone (PTH). PTH Analog includes free base, pharmaceutically acceptable salts, pharmacologically active metabolites of PTH Analog and their pharmaceutically acceptable salts, hydrates, its racemates, its enantiomers or complexes. PTH analog as per present invention includes teriparatide and abaloparatide.

The term “teriparatide” used herein includes free base, pharmaceutically acceptable salts, pharmacologically active metabolites of teriparatide and their pharmaceutically acceptable salts, hydrates, its racemates, its enantiomers or complexes. The teriparatide or salt or complex thereof to be used is in an amount equivalent to about 0.5 mg to about 15 mg of teriparatide free base.

The term “abaloparatide” used herein includes free base, pharmaceutically acceptable salts, pharmacologically active metabolites of abaloparatide and their pharmaceutically acceptable salts, hydrates, its enantiomers or its racemates unless otherwise noted. The abaloparatide or salt or complex thereof to be used is in an amount equivalent to about 80 mg to about 8000 mg of abaloparatide free base.

“Therapeutically effective amount” or “effective amount” refers to the amount of a pharmaceutically active agent when administered to a patient for treating a disease, is sufficient to affect such prevention or treatment for the disease. The “therapeutically effective amount” will vary depending on the disease and its severity, and the age, weight, and other conditions of the patient to be treated.

The term “oral pharmaceutical compositions” herein refers to any composition which comprises PTH analogs for oral administration includes but are not limited to immediate release, delayed release, extended release and pulsed-release.

Relative bioavailability refers to bioavailability measured using below formula

F %=(AUC test drug/AUC reference)×(Dose reference Dose test drug)×100).

The term “degradation preventing agents” refers to one or more agents that prevents enzymatic degradation of PTH analogue.

The term “metal containing compound” means any substance, complex, salts containing at least one metal ion. The term “vanadium containing compound” means any substance, complex, salts containing vanadium in any form. The term “chromium containing compound” means any substance, complex, salts containing chromium in any form. The term “manganese containing compound” means any substance, complex, salts containing manganese in any form.

The term “reducing agent” means a substance that reduces a chemical compound usually by donating electrons in a redox chemical reaction.

In one embodiment, the present invention relates to a method of preventing or treating disease or disorder comprising administration of oral pharmaceutical composition comprising therapeutically effective amount of PTH analog to a patient in need thereof, wherein said composition provides relative bioavailability of at least 0.5%, compare to subcutaneous administration. In a similar embodiment, invention relates to administration of oral pharmaceutical composition comprising therapeutically effective amount of teriparatide or abaloparatide to a patient in need thereof.

In another embodiment invention relates to a method of preventing or treating disease or disorder comprising administration of oral pharmaceutical composition comprising therapeutically effective amount of PTH analog to a patient in need thereof, wherein said oral pharmaceutical composition achieves maximum plasma concentration (Tmax) between 45 min and 90 min. In a similar embodiment, invention relates to administration of oral pharmaceutical composition comprising therapeutically effective amount of teriparatide or abaloparatide to a patient in need thereof.

In one embodiment, the present invention relates to a method of preventing or treating disease or disorder comprising administration of oral pharmaceutical composition comprising therapeutically effective amount of PTH analog, to a patient in need thereof, wherein said composition provides relative bioavailability of at least 0.5% compare to subcutaneous administration and wherein said oral pharmaceutical composition further comprises at least one degradation preventing agent. In a similar embodiment, invention relates to administration of oral pharmaceutical composition comprising therapeutically effective amount of teriparatide or abaloparatide to a patient in need thereof. In another embodiment, at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof. In one or more embodiment, pharmaceutical composition optionally one or more selected from group consisting of absorption enhancer, Pgp efflux inhibitor. In another embodiment, PTH analogue and degradation preventing agent are present in physically separated form in said pharmaceutical composition. In another embodiment, PTH analogue is in enteric coated form and degradation preventing agent is in enteric coated form in said pharmaceutical composition.

In one embodiment, the present invention relates to an oral pharmaceutical composition comprising therapeutically effective amount of PTH analog, wherein said composition upon oral administration provides relative bioavailability of at least 0.5%, compare to subcutaneous administration. In a similar embodiment, invention relates to an oral pharmaceutical composition comprising therapeutically effective amount of teriparatide or abaloparatide, wherein said composition upon oral administration provides relative bioavailability of at least 0.5%, compare to subcutaneous administration.

In another embodiment invention relates to an oral pharmaceutical composition comprising therapeutically effective amount of PTH analog, wherein said oral pharmaceutical composition upon oral administration provides maximum plasma concentration (Tmax) between 45 min and 90 min. In a similar embodiment, invention relates to an oral pharmaceutical composition comprising therapeutically effective amount of teriparatide or abaloparatide, wherein said oral pharmaceutical composition upon oral administration provides maximum plasma concentration (Tmax) between 45 min and 90 min.

In one embodiment, the present invention relates to an oral pharmaceutical composition comprising therapeutically effective amount of PTH analog, wherein said composition upon oral administration provides relative bioavailability of at least 0.5% compare to subcutaneous administration and wherein said oral pharmaceutical composition further comprises at least one degradation preventing agent. In a similar embodiment, invention relates to an oral pharmaceutical composition comprising therapeutically effective amount of teriparatide or abaloparatide, wherein said composition upon oral administration provides relative bioavailability of at least 0.5% compare to subcutaneous administration and wherein said oral pharmaceutical composition further comprises at least one degradation preventing agent. In another embodiment, at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof. In one or more embodiment, pharmaceutical composition optionally one or more selected from group consisting of absorption enhancer, Pgp efflux inhibitor. In another embodiment, PTH analogue and degradation preventing agent are present in physically separated form in said pharmaceutical composition. In another embodiment, PTH analogue is in enteric coated form and degradation preventing agent is in enteric coated form in said pharmaceutical composition.

### PTH Analog

PTH Analog/s are the active substance having at least partial structural similarity with PTH and acts at least partially similar to parathyroid hormone (PTH). PTH analogues are anabolic agents developed to enhance uptake of calcium, stimulate new bone formation and reduces risk of osteoporosis fracture. PTH analog as per present invention includes teriparatide and abaloparatide.

Teriparatide, also called PTH (1-34), is human parathyroid hormone (1-34). It has an identical sequence to the 34 N-terminal amino acids of the 84-amino acid human parathyroid hormone which is the biologically active region of PTH. Currently, it is commercially available as multi-dose prefilled delivery pen for subcutaneous administration (FORTEO by Eli Lilly). Forteo contains teriparatide prepared by recombinant DNA technology. Teriparatide is currently approved for the treatment of following indications in dosage of 20 mcg of teriparatide per dose each day:
a. Treatment of Postmenopausal Women with Osteoporosis at High Risk for Fracture.
b. Increase of Bone Mass in Men with Primary or Hypogonadal Osteoporosis at High Risk for Fracture.
c. Treatment of Men and Women with Glucocorticoid Induced Osteoporosis at High Risk for Fracture.

Abaloparatide is an analog of human parathyroid hormone related peptide, PTHrP(1-34). It has 41% homology to hPTH(1-34) (human parathyroid hormone 1-34) and 76% homology to hPTHrP(1-34) (human parathyroid hormone-related peptide 1-34). Abaloparatide is currently approved for the treatment of for the treatment of postmenopausal women with osteoporosis at high risk for fracture, in dosage of 80 mcg subcutaneously once daily. It is currently available as pre-assembled single patient use disposable pen for subcutaneous administration.

The PTH analog or salt or complex thereof to be used is in an amount equivalent to about 0.5 mg to about 15 mg of PTH analog free base. The teriparatide or salt or complex thereof to be used is in an amount equivalent to about 0.5 mg to about 15 mg of teriparatide free base. The abaloparatide or salt or complex thereof to be used is in an amount equivalent to about 80 mg to about 800 mg of teriparatide free base.

### Relative Bioavailability

Relative bioavailability refers to bioavailability measured using below formula F %=(AUC test drug/AUC reference)×(Dose reference/Dose test drug)×100). In one or more embodiment, invention relates to method of preventing or treating disease or disorder comprising administration of oral pharmaceutical composition of PTH analog to a patient in need thereof, wherein said composition provides relative bioavailability of at least 0.5%, 0.5-10%, 1-9%, 2-8%, 3-7% or 4-6% compare to subcutaneous administration.

### Concurrent Administration of Antiresorptive Agent

In one embodiment present invention comprises concurrent administration of at least one Antiresorptive agent. Antiresorptive agent includes compound selected from bisphosphonate (e.g. Ibandronate, Pamidronate, Alendronate, Zoledronic acid Risedronate, Tiludronate, Etidronate, Minodronate), Selective estrogen receptor modulators Raloxifene, Lasofoxifene, Bazodoxifene, Arzoxifene, Ospemifene, calcitonin, Vitamin D, or mixture thereof.

### Degradation Preventing Agents

Degradation preventing agents refers to one or more agents that prevents enzymatic degradation of proteins or peptides. In one or more embodiment, degradation preventing agents is combination of metal containing compound and reducing agent. In one or more embodiment, degradation preventing agents is combination of vanadium containing compound, chromium containing compound or manganese containing compound, and reducing agent.

### Metal Containing Compounds

In one or more embodiments, metal containing compound refers to any substance, complex, salts containing at least one metal ion. In one or more embodiment, metal containing compound is selected from group consisting of is vanadium containing compound, chromium containing compound or manganese containing compound.

### Vanadium Containing Compounds

Vanadium containing compound refers to any substance, complex, salts containing vanadium in any form. Vanadium containing compound is in an amount equivalent to 0.5 mg to 15 mg of vanadium. In one or more embodiment, Vanadium containing compound is selected from group consisting of vanadium (V) oxide, sodium vanadate, vanadium sulfate, vanadyl sulfate, vanadium biguanide, bis(maltolato)oxavanadium (IV), vanadium acetate, vanadyl picolinate and vanadyl citrate. In one or more embodiment, Vanadium containing compound is vanadium sulphate.

### Chromium Containing Compounds

Chromium containing compound refers to any substance, complex, salts containing chromium in any form. Chromium containing compound is in an amount equivalent to 0.1 mg to 3 mg of chromium. In one or more embodiment, Chromium containing compound is selected from group consisting of chromium picolinate, chromium polynicotinate, chromium nicotinate, chromium chloride and chromium acetate. In one or more embodiment, Chromium containing compound is chromium picolinate.

### Manganese Containing Compounds

Manganese containing compound refers to any substance, complex, salts containing manganese in any form. Manganese containing compound is in an amount equivalent to 0.5 mg to 5 mg of manganese. In one or more embodiment, Manganese containing compound is selected from group consisting of manganese gluconate, manganese sulfate, potassium permanganate and manganese chloride. In one or more embodiment, Manganese containing compound is manganese gluconate.

### Reducing Agents

Reducing agent refers to a substance that reduces a chemical compound usually by donating electrons in a redox chemical reaction. In an embodiment, the at least one reducing agent is selected from any or a combination of ascorbic acid, reduced glutathione, cysteine, uric acid, reducing sugar, glyceraldehyde, α-tocopherol, vitamin A, α-lipoic acid, dihydro-α-lipoic acid, glucose, galactose, lactose, maltose, thiol-bearing compound, a thiomer and pharmaceutically acceptable salts thereof. In an embodiment, the pharmaceutical composition includes the at least one reducing agent in an amount ranging from about 1 mg to about 1000 mg per unit dose.

### Absorption Enhancers

The terms “absorption enhancer” and “permeation enhancer” as interchangeably and synonymously used herein throughout the present disclosure encompass within its meaning, absorption enhancers and permeation enhancers, as known to or appreciated by a person skilled in the pertinent art. In an embodiment, administration of least one absorption or permeation enhancer improves or facilitates the mucosal absorption of the PTH analog in the gastrointestinal tract. In an embodiment, the at least one absorption or permeation enhancer is selected from any or a combination of zwitter-ionic absorption enhancer or a non-ionic absorption enhancer. In an embodiment, the at least one absorption enhancer is selected from any or a combination of C8-20 alkanoyl carnitine (preferably lauroyl carnitine, myristoylcarnitine or palmitoyl carnitine; e.g., lauroyl carnitine chloride, myristoyl carnitine chloride or paimitoyi carnitine chloride), salicylic acid (preferably a salicylate, e.g., sodium salicylate), a salicylic acid derivative (such as 3-methoxysalicylicacid, 5-methoxysalicylic acid, or homovanillic acid, a C8-20 alkanoic acid (preferably a C8-20 alkanoate, more preferably a caprate, a caprylate, a myristate, a palmitate, or a stearate, such as sodium caprate, sodium caprylate, sodiummyristate, sodium palmitate, or sodium stearate), citric acid (preferably a citrate such as sodium citrate), sodium lauroyl alaninate, Ndodecanoyl-L-alanine, sodiumlauroyl asparaginate, N-dodecanoyl-L-asparagine, sodium lauroyl aspartic acid, N-dodecanoyl-L-aspartic acid, sodiumlauroyl cysteinate, N-dodecanoyl-L-cysteine, sodium lauroyl glutamic acid, N-dodecanoyl-L-glutamic acid, sodium lauroylglutaminate, N-dodecanoyl-L-glutamine, sodium lauroyl glycinate, N-dodecanoyl-Lglycine, sodium lauroyl histidinate,N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, Ndodecanoyl-L-phenylalanine, sodium lauroyl prolinate, N-dodecanoyl-L-proline, sodium lauroyl serinate, N-dodecanoyl-Lserine, sodium lauroyl threoninate, N-dodecanoyl-Lthreonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, sodiumlauroyl sarcosinate, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, sodium capric cysteinate, N-decanoyl-L-cysteine, sodium capric glutamic acid, N-decanoyl-L-glutamic acid, sodium capric glutaminate, N-decanoyl-Lglutamine, sodium capric glycinate, N-decanoyl-L-glycine, sodium capric histidinate, Ndecanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate, Ndecanoyl-L-proline, sodium capric serinate, N-decanoyl-L-serine, sodium capric threoninate, N-decanoyl-Lthreonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, N-decanoyi-L-sarcosine, sodium oleoyl sarcosinate, sodium Ndecylleucine, sodium stearoyl glutamate (Amisoft HS-11 P), sodium myristoyl glutamate (Amisoft MS-11), sodium lauroyl glutamate (Amisoft LS-11), sodium cocoyl glutamate (Amisoft CS-11), sodiumcocoyl glycinate (Amilite GCS-11), sodium N-decyl leucine, sodium cocoyl glycine, sodium cocoyl glutamate, sodium lauroyl alaninate, N-dodecanoyl-Lalanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamicacid, N-dodecanoyl-L-glutamic acid, sodium lauroyl glutaminate, Ndodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyi-Lisoleucine, sodium lauroyl leucinate, N-dodecanoyl-L-leucine, sodium lauroyl methinoninate, N-dodecanoyl-L-methionine, sodium lauroyl phenylalaninate, N-dodecanoyl-Lphenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodiumlauroyl tryptophanate, N-dodecanoyl-L-tryptophane, sodium lauroyl tyrosinate, Ndodecanoyl-L-tyrosine, sodiumlauroyl valinate, N-dodecanoyl-L-valine, N-dodecanoyl-Lsarcosine, sodium capric alaninate, N-decanoyl-L-alanine, sodiumcapric asparaginate, Ndecanoyl-L-asparagine, sodium capric aspartic acid, N-decanoyl-L-aspartic acid, Sodium capric cysteinate, N-decanoyl-L-cysteine, sodium capric glutamic acid, N-decanoyl-Lglutamic acid, sodium capricglutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-Lleucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-Lthreonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and the pharmaceutically acceptable salts of any of the aforementioned compounds such as C8-20 alkanoyl sarcosinate (a lauroyl sarcosinate, such as sodium lauroyl sarcosinate) or one of the 20 standard proteinogenic α-amino acids that is acylated with a C8-20 alkanoic acid), an alkylsaccharide (C1-20 alkylsaccharide such as C8-10 alkylpolysaccharide like Multitrope™ 1620-LQ-(MV), or n-octyl-beta-Dglucopyranoside, or n-dodecyl-beta-D-maltoside), a cyclodextrine (α-cyclodextrin, β-cyclodextrin, γ-cyclodextrin, methyl-β-cyclodextrin, hydroxypropyl β-cyclodextrin, or sulfobutylether β-cyclodextrin), sodium N-[8-(2-hydroxybenzoyl)amino]caprylate (SNAC), a thiomer (includes the thiomers that are disclosed in Laffleur F et al., Future Med Chem. 2012, 4, 2205-16), a calcium chelating compound (ethylenediaminetetraacetic acid (EDTA), ethylene glycol tetraacetic acid (EGTA), sodium citrate, or polyacrylic acid), cremophor EL (Kolliphor EL; CAS no. 61791-12-6), chitosan, N,N,N-trimethyl chitosan, benzalkonium chloride, bestatin, cetylpyridinium chloride, cetyltrimethylammonium bromide, a C2-20 alkanol (e.g., ethanol, decanol, lauryl alcohol, myristyl alcohol, or palmityl alcohol), a C8-20 alkenol (e.g., oleyl alcohol), a C8-20 alkenoic acid (e.g., oleic acid), dextran sulfate, diethyleneglycol monoethyl ether (transcutol), 1-dodecylazacyclo-heptan-2-one (Azone®), ethyl caprylate, glyceryl monolaurate, lysophosphatidylcholine, menthol, a C8-20 alkylamine, a C8-20 alkenylamine (e.g., oleylamine), phosphatidylcholine, a poloxamer, polyethylene glycol monolaurate, polyoxyethylene, polypropylene glycol monolaurate, a polysorbate (polysorbate 80), a deoxycholate (sodium deoxycholate), sodium glycocholate, sodium glycodeoxycholate, sodium lauryl sulfate (SDS), a taurocholate (e.g., sodium taurocholate), a taurodeoxycholate (sodium taurodeoxycholate), sucrose laurate, a sulfoxide (a (C1-10 alkyl)-(C1-10alkyl)-sulfoxide, such as, decyl methyl sulfoxide, or dimethyl sulfoxide), cyclopentadecalactone, 8-(N-2-hydroxy-5-chloro-benzoyl)-amino-caprylic acid (5-CNAC), dodecyl-2-N,N-dimethylamino propionate (DDAIP), D-α-tocopheryl polyethylene glycol-1000 succinate (TPGS), and pharmaceutically acceptable salts of the aforementioned compounds and the likes. In an embodiment, a mixture of any of two or more absorption enhancers, including the above-described absorption enhancers, can be used. However, any or a combination of absorption enhancer(s), as known to or appreciated by a person skilled in the art, can be utilized to serve its intended purpose, as laid in the present disclosure, without departing from the scope and spirit of the present invention.

### Pgp Efflux Inhibitors

P-glycoprotein is an efflux transporter that transports drug molecule from cell cytoplasm to intestinal lumen for excretion. These transporters are found on luminal side of the enterocytes in the small intestine. It limits the bioavailability of some orally administered drugs. In one or more embodiments pgp efflux inhibitors is selected from group consisting of naringin, piperine, bergamottin, quercetin, quinidine, quinine, reserpine, ritonavir, tariquidar, and verapamil.

### Pharmaceutical Composition

The term “oral pharmaceutical compositions” herein refers to any composition which comprises PTH analogs for oral administration includes but are not limited to immediate release, delayed release, extended release and pulsed-release.

In one or more embodiments, pharmaceutical composition comprising therapeutically effective amount of PTH analogue and said at least one degradation preventing agent are present in physically separated form in said pharmaceutical composition.

In one or more embodiments, pharmaceutical composition comprises therapeutically effective amount of PTH analogue in enteric coated form and combination of metal containing compound and reducing agent in enteric coated form.

Oral pharmaceutical compositions may be prepared by any conventional techniques including but not limited to dry granulation, wet granulation, melt granulation, direct compression, extrusion-spheronization or compression coating.

In an embodiment, an oral pharmaceutical dosage form is selected from any or a combination of tablets (coated or uncoated tablets), capsules (soft gelatin capsules, hard gelatin capsules, HPMC capsules, or HPMCP capsules), a capsule-in-capsule, tablet-in capsule, lozenges, troches, ovules, solutions, emulsions, suspensions, syrups, elixirs, powders and granules for reconstitution, dispersible powders and granules, medicated gums, chewing tablets, effervescent tablets, multi-particulate dosage forms and the likes. However, any or a combination of oral pharmaceutical dosage form(s), as known to or appreciated by a person skilled in the art, can be utilized to serve its intended purpose, as laid in the present disclosure, without departing from the scope and spirit of the present invention.

In an embodiment, the pharmaceutical composition further includes optionally any or a combination of one or more pharmaceutically acceptable excipients, such as but not limited to carriers, diluents, fillers, disintegrants, lubricating agents, binders, colorants, pigments, stabilizers, preservatives, antioxidants, and/or solubility enhancers. In an embodiment, the pharmaceutical composition, optionally, further includes one or more pharmaceutically acceptable additives such as vitamin E, histidine, microcrystalline cellulose (MCC), mannitol, starch, sorbitol and/or lactose. In an embodiment, the pharmaceutical compositions can be formulated by any techniques known to or appreciated by a person skilled in the art, to serve its intended purpose, as laid in the present disclosure, without departing from the scope and spirit of the present invention.

In an embodiment, the at least one solubility enhancers is selected from any or a combination of poly(ethylene glycol), including poly(ethylene glycol) having a molecular weight in the range of about 200 to about 5,000 Da, ethylene glycol, propylene glycol, nonionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-α-cyclodextrin, hydroxyethyl-γ-cyclodextrin, hydroxypropyl-γ-cyclodexin, dihydroxypropyl-β-cyclodextrin, Sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate and the likes.

### Enteric Coating:

Composition of the any embodiment of the invention may be enteric coated using any known method. Enteric polymers include one or more of hydroxypropyl methylcellulose phthalate; polyvinyl acetate phthalate; hydroxypropylmethylcellulose acetate succinate; alginate; carbomer; carboxymethyl cellulose; methacrylic acid copolymer; shellac; cellulose acetate phthalate; starch glycolate; polacrylin; cellulose acetate phthalate; methyl cellulose acetate phthalate; hydroxypropylcellulose acetate phthalate; cellulose acetate terephthalate; cellulose acetate isophthalate; and cellulose acetate trimellitate. Composition may also be prepared using readymade capsule made of enteric polymers and such composition be also considered as enteric coated forms.

### Examples

| | Formulation T1 (MIRA 5 Capsule) | |
|---|---|---|
| Sr No | Material | Quantity per capsule (mg) |
| 1 | Ascorbate sodium | 30 |
| 2 | Vanadium Sulfate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T1 (PA + Teriparatide Capsule) | |
| Sr No | Material | Quantity per capsule (mg) |
| 1 | Teriparatide | 0.5 |
| 2 | Labrasol ALF | 5 |
| 3 | Microcrystalline cellulose 101 | 17 |
| 4 | Mannitol | 5 |
| 5 | Croscarmellose sodium | 2.5 |
| 6 | HPMC E5 (Binder) | QS |
| | Total | 30 |

| | Formulation T2 (MIRA 2 Capsule) | |
|---|---|---|
| Sr No | Material | Quantity per capsule (mg) |
| 1 | Ascorbate sodium | 30 |
| 2 | Vanadium oxide | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T2 (PA + Teriparatide Capsule) | |
| Sr No | Material | Quantity per capsule (mg) |
| 1 | Teriparatide | 0.5 |
| 2 | Labrasol ALF | 5 |
| 3 | Microcrystalline cellulose 101 | 17 |
| 4 | Mannitol | 5 |
| 5 | Croscarmellose sodium | 2.5 |
| 6 | HPMC E5 (Binder) | QS |
| | Total | 30 |

| | Formulation T3 (MIRA 5 Capsule) | |
|---|---|---|
| Sr No | Material | Quantity per capsule (mg) |
| 1 | Ascorbate sodium | 30 |
| 2 | Vanadium sulfate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T3 (PA + Teriparatide Capsule) | |
| Sr No | Material | Quantity per capsule (mg) |
| 1 | Teriparatide | 0.5 |
| 2 | Naringin | 5 |
| 3 | Labrasol ALF | 3 |
| 4 | Microcrystalline cellulose 101 | 14 |
| 5 | Mannitol | 5 |
| 6 | Croscarmellose sodium | 2.5 |
| 7 | HPMC E5 (Binder) | QS |
| | Total | 30 |

| | Formulation T4 (MIRA 3 Capsule) | |
|---|---|---|
| Sr No | Material | Quantity per capsule (mg) |
| 1 | Uric acid | 3 |
| 2 | Sodium Vanadate | 0.3 |
| 3 | Microcrystalline cellulose 101 | 14 |
| 4 | Mannitol | 5.2 |
| 5 | Croscarmellose sodium | 2.5 |
| 6 | HPMC E5 (Binder) | QS |
| | Total | 25 |
| | Formulation T4 (PA + Teriparatide Capsule) | |
| Sr No | Material | Quantity per capsule (mg) |
| 1 | Teriparatide | 0.5 |
| 2 | Piperine | 3 |
| 3 | Poloxamer (Pluronic F 168) | 5 |
| 4 | Microcrystalline cellulose 101 | 14 |
| 5 | Mannitol | 5 |
| 6 | Croscarmellose sodium | 2.5 |
| 7 | HPMC E5 (Binder) | QS |
| | Total | 30 |

| | Formulation T5 (MIRA 5 Capsule) | |
|---|---|---|
| Sr No | Material | Quantity per capsule (mg) |
| 1 | Ascorbate sodium | 30 |
| 2 | Vanadium sulfate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T5 (PA + Teriparatide Capsule) | |
| Sr No | Material | Quantity per capsule (mg) |
| 1 | Teriparatide | 0.5 |
| 2 | Piperine | 3 |
| 3 | Labrasol ALF | 5 |
| 4 | Microcrystalline cellulose 101 | 14 |
| 5 | Mannitol | 5 |
| 6 | Croscarmellose sodium | 2.5 |
| 7 | HPMC E5 (Binder) | QS |
| | Total | 30 |

| | Formulation T6 (MIRA 4 Capsule) | |
|---|---|---|
| Sr No | Material | Quantity per capsule (mg) |
| 1 | Ascorbate sodium | 30 |
| 2 | Manganese gluconate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T6 (PA + Teriparatide Capsule) | |
| Sr No | Material | Quantity per capsule (mg) |
| 1 | Teriparatide | 0.25 |
| 2 | Labrasol ALF | 5 |
| 3 | Microcrystalline cellulose 101 | 17 |
| 4 | Mannitol | 5.25 |
| 5 | Croscarmellose sodium | 2.5 |
| 6 | HPMC E5 (Binder) | QS |
| | Total | 30 |

| | Formulation T7 (MIRA 5 Capsule) | |
|---|---|---|
| Sr No | Material | Quantity per capsule (mg) |
| 1 | Ascorbate sodium | 30 |
| 2 | Vanadium sulfate | 0.3 |
| 3 | Reduced Glutathione | 12 |
| 4 | Chromium Picolinate | 0.3 |
| 6 | Microcrystalline cellulose 101 | 4.9 |
| 7 | Croscarmellose sodium | 2.5 |
| 8 | HPMC E5 (Binder) | QS |
| | Total | 50 |
| | Formulation T7 (PA + Teriparatide Capsule) | |
| Sr No | Material | Quantity per capsule (mg) |
| 1 | Teriparatide | 0.5 |
| 2 | Labrasol ALF | 5 |
| 5 | Microcrystalline cellulose 101 | 17 |
| 6 | Mannitol | 5 |
| 7 | Croscarmellose sodium | 2.5 |
| 8 | HPMC E5 (Binder) | QS |
| | Total | 30 |

| | Formulation T8 (MIRA 1 Capsule) | |
|---|---|---|
| Sr No | Material | Quantity per capsule (mg) |
| 1 | Reduced Glutathione | 12 |
| 2 | Chromium Picolinate | 0.3 |
| 3 | Microcrystalline cellulose 101 | 10 |
| 4 | Mannitol | 5.2 |
| 5 | Crocarmellose sodium | 2.5 |
| 6 | HPMC E5 (Binder) | QS |
| | Total | 30 |
| | Formulation T8 (PA + Teriparatide Capsule) | |
| Sr No | Material | Quantity per capsule (mg) |
| 1 | Teriparatide | 0.5 |
| 2 | Labrasol ALF | 5 |
| 3 | Ascorbate sodium | 30 |
| 4 | Vanadium oxide | 0.3 |
| 5 | Microcrystalline cellulose 101 | 11.7 |
| 7 | Croscarmellose sodium | 2.5 |
| 8 | HPMC E5 (Binder) | QS |
| | Total | 50 |

| | Formulation T9 (MIRA 5 Capsule) | |
|---|---|---|
| Sr No | Material | Quantity per capsule (mg) |
| 1 | Ascorbate sodium | 30 |
| 2 | Vanadium Sulfate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T9 (PA + Teriparatide Capsule) | |
| Sr No | Material | Quantity per capsule (mg) |
| 1 | Teriparatide | 0.5 |
| 2 | Solutol HS 15 | 5 |
| 3 | Microcrystalline cellulose 101 | 17 |
| 4 | Mannitol | 5 |
| 5 | Croscarmellose sodium | 2.5 |
| 6 | HPMC E5 (Binder) | QS |
| | Total | 30 |

| | Formulation T10 (MIRA 5 Capsule) | |
|---|---|---|
| Sr No | Material | Quantity per capsule (mg) |
| 1 | Ascorbate sodium | 30 |
| 2 | Vanadium Sulfate | 0.3 |
| 3 | Microcrystalline cellulose 101 | 12 |
| 4 | Mannitol | 5.2 |
| 5 | Croscarmellose sodium | 2.5 |
| 6 | HPMC E5 (Binder) | QS |
| | Total | 50 |
| | Formulation T10 (PA + Teriparatide Capsule) | |
| Sr No | Material | Quantity per capsule (mg) |
| 1 | Teriparatide | 0.5 |
| 2 | Vit E (TPGS) | 5 |
| 3 | Microcrystalline cellulose 101 | 17 |
| 4 | Mannitol | 5 |
| 5 | Croscarmellose sodium | 2.5 |
| 6 | HPMC E5 (Binder) | QS |
| | Total | 30 |

### Process of Preparation:

### Granulation Procedure

### A. Preparation of Binder Solution:

0.3% W/V of HPMC E-5 solution was prepared by dissolving 75.0 mg of HPMC E-5 in 25.0 mL of De-ionized (D.I.) water.

### B. Preparation of Powder Blend:

All the ingredients except Liquid excipients were weighed accurately and mixed in polybag for 5.0 minutes.

### C. Addition of Binder:

Weighed quantity of Liquid excipients along with peptide was added in HPMC E-5 (0.03%) binder solution. Resulting mixture was added dropwise to perform wet granulation.

### D. Drying of Granules:

Granules were dried in a vacuum desiccator over silica bed overnight.

### E. Sifting of Granules:

Dried granules were passed through a stainless steel 40 # mesh, collected in a suitable glass container and stored at room temperature.

### Study Procedure

To quantify teriparatide level in rat plasma using ELISA kit after dosing several formulations of teriparatide to distal small intestine (ileum) in S.D.rat.
Reference formulation was dosed at 10 mcg per subject into the tail (n=3). Test formulations was dosed at follows in N=3 subjects each.
1 Capsule labeled with MIRA was triturated in a mortar & pestle, transferred to eppendorf & mixed in 2 ml/kg of tris buffer & dosed to one animal to it's ileum via catheter. 5 minutes later, one capsule labeled with PA was triturated in a mortar & pestle, transferred to appendorf & mixed in 2 ml/kg of tris buffer & dosed to same animal to it's ileum via catheter.
One capsule labeled with MIRA was triturated in a mortar & pestle (kept on a ice box), transferred to eppendorf & mixed in 2 ml/kg of tris buffer & dosed to one animal to its ileum via catheter.
minutes later one capsule of API plus PA was triturated in a mortar & pestle (kept on a ice box), transferred to appendorf & mixed in 2 ml/kg of tris buffer & dosed to same animal to it's ileum via catheter.
˜100 μl of blood was collected in a pre-filled Na-EDTA eppendorf, from retro-orbital sinus puncture using glass capillaries. Blood samples were centrifuged at 5000 rpm, 5 min, 4° c. to obtain plasma. Plasma samples were stored at −80 till analysis. During dosing, the blood collection time points were at 0, 5, 10, 15, 20, 30, 60- and 120-min post dose. ˜100 μl of blood was collected in a pre-filled Na-EDTA eppendorf, from retro-orbital sinus puncture. Blood was centrifuged at 5000 rpm, 5 min, 4° c. to obtain plasma.

### Study Results

| | Tmax | Cmax | % Relative |
|---|---|---|---|
| Group | (min) | (ng/mL) | bioavailability |
| Formulation I | 15.00 | 4,494 | |
| (Teriparatide) | | | |
| Formulation T1 | 35.00 | 2,853 | 2.36 |
| Formulation T2 | 15.00 | 2,752 | 1.05 |
| Formulation T3 | 15.00 | 2,184 | 1.59 |
| Formulation T4 | 5.00 | 968 | 0.22 |
| Formulation T5 | 13.33 | 2,612 | 1.08 |
| Formulation T6 | 15.00 | 3,335 | 3.53 |
| Formulation T7 | 6.67 | 2,299 | 0.97 |
| Formulation T8 | 16.67 | 2,817 | 3.64 |
| Formulation T9 | 31.67 | 3,278 | 3.18 |
| Formulation T10 | 13.33 | 2,868 | 2.39 |

### Formulation T11

| | | | |
|---|---|---|---|
| Sr. No. | Ingredient name | Technical grade | mg/Unit |

Inner capsule composition

| | | | |
|---|---|---|---|
| 1 | Teriparatide acetate | API | 4.08 |
| 2 | Macrogol 15 Hydroxystearate, | Kolliphore HS 15 | 20.00 |
| | Polyoxyl 15 Hydroxystearate | | |
| 3 | Microcrystalline cellulose | Avicel PH 101 | 74.92 |
| 4 | Mannitol | Pearlitol 160C | 16.00 |
| 5 | Croscarmellose sodium | Ac-Di-sol SD 711 | 9.00 |
| 6 | Hypromellose | Pharmacoat 606 | 6.00 |
| | Net fill weight in inner capsule | | 130.00 |
| | Hard gelatin Capsule size 3 | | |

Outer capsule composition

| | | | |
|---|---|---|---|
| 7 | Ascorbic acid | — | 100.00 |
| 8 | Vanadium sulfate | — | 10.00 |
| 9 | Microcrystalline cellulose | Avicel PH 102 | 25.00 |
| 10 | Mannitol | Pearlitol 160C | 7.00 |
| 11 | Croscarmellose Sodium | Ac-Di-sol SD 711 | 8.00 |
| | Net fill weight in Outer Capsule | | 150.00 |

### Manufacturing Process:

### Part I: Inner Capsule

1. All the inactive ingredients from Sr. No. 2 to 6 were weighed accurately.
2. Microcrystalline cellulose, Mannitol, Hypromellose were sifted through #40 mesh.
3. Granulation solvent preparation: Required quantity of purified water was weighed into glass beaker followed by dissolution of Kolliphore HS 15 in it under continuous mixing using glass rod till clear solution formed.
4. Prepared solution was added dropwise in mixture from step 2 to reach granulation end point by manual mixing.
5. Resulting wet mass was dried in hot air oven at 50° C.
6. The dried granules were sifted through #40 mesh.
7. Croscarmellose sifted through #40 and added to step 6 by geometric mixing in the polybag.
8. The granular blend was weighed in order to mix with required quantity of API (teriparatide) with necessary precautions. Co-sifting of API along with base granules was done through #40 mesh.
9. The obtained blend was mixed manually in polybag to ensure uniformity in content followed by sifting through #40.
10. The capsules (Size ‘3’) were filled with target weight 130.0 mg.

### Part II: Inner Capsule

11. All the ingredients from Table (Sr. No 7 to 11) were weighed accurately. These ingredients were sifted through #40 mesh and mixed in a poly bag.
12. Size ‘3’ filled capsules from step 10 were incorporated in Size ‘0’ enteric capsules.
13. After Step 12 the blend prepared in step 11 was accommodated in size 0 with net fill weight as 150 mg.
14. Packing: Capsules are packed in HDPE containers, nitrogen gas was purged inside before closing the container.

| Dissolution | Time | | % Release |
|---|---|---|---|
| 0.1N HCL, 500 mL, 75 RPM (ACID STAGE) | 2 | Hr | 0 |
| 6.8 Phosphate Buffer, 500 mL, 150 RPM | 15 | min | 18 |
| (Buffer Stage) | 30 | min | 82 |
| | 60 | min | 84.0 |

### Formulation T12

| | | | |
|---|---|---|---|
| S. No. | Ingredient name | Technical grade | mg/Unit |

Inner capsule composition

| | | | |
|---|---|---|---|
| 1 | Teriparatide acetate | API | 4.08 |
| 2 | Caprylocaproyl Polyoxyl-8 | Labrasol ALF | 20.00 |
| | glycerides | | |
| 3 | Microcrystalline cellulose | Avicel PH 101 | 74.92 |
| 4 | Mannitol | Pearlitol 160C | 12.00 |
| 5 | Croscarmellose sodium | Ac-Di-sol SD 711 | 9.00 |
| 6 | Hypromellose | Pharmacoat 606 | 10.00 |
| | Net fill weight in inner capsule | | 130.00 |
| | Hard gelatin Capsule size 3 | | |

Outer capsule composition

| | | | |
|---|---|---|---|
| 7 | Ascorbic acid | — | 100.00 |
| 8 | Manganese gluconate | — | 3.00 |
| 9 | Microcrystalline cellulose | Avicel PH 102 | 30.00 |
| 10 | Mannitol | Pearlitol 160C | 8.00 |
| 11 | Croscarmellose Sodium | Ac-Di-sol SD 711 | 9.00 |
| | Net fill weight in Outer Capsule | | 150.00 |

### Manufacturing Process:

### Part I: Inner Capsule

1. All the inactive ingredients from Sr. No. 2 to 6 were weighed accurately
2. Microcrystalline cellulose, Mannitol, Hypromellose were sifted through #40 mesh.
3. Granulation solvent preparation: Required quantity of purified water was weighed into glass beaker followed by dissolution of Labrasol ALF in it under continuous mixing using glass rod till clear solution formed.
4. Prepared solution was added dropwise in mixture from step 2 to reach granulation end point by manual mixing.
5. Resulting wet mass was dried in hot air oven at 50° C.
6. The dried granules were sifted through #40 mesh.
7. Croscarmellose sifted through #40 and added to step 6 by geometric mixing in the polybag.
8. The granular blend was weighed in order to mix with required quantity of API (teriparatide) with necessary precautions. Co-sifting of API along with base granules was done through #40 mesh.
9. The obtained blend was mixed manually in polybag to ensure uniformity in content followed by sifting through #40.
10. The capsules (Size ‘3’) were filled with target weight 130.0 mg.

### Part II: Inner Capsule

11. All the ingredients from Table (Sr. No 7 to 11) were weighed accurately. These ingredients were sifted through #40 mesh and mixed in a poly bag.
12. Size ‘3’ filled capsules from step 10 were incorporated in Size ‘0’ enteric capsules.
13. After Step 12 the blend prepared in step 11 was accommodated in size 0 with net fill weight as 150 mg.
14. Packing: Capsules are packed in IDPE containers, nitrogen gas was purged inside before closing the container.

| Dissolution | Time | | % Release |
|---|---|---|---|
| 0.1N HCL, 500 mL, 75 RPM (ACID STAGE) | 2 | Hr | 0 |
| 6.8 Phosphate Buffer, 500 mL, 150 RPM | 15 | min | 46 |
| (Buffer Stage) | 30 | min | 85 |
| | 60 | min | 96 |

We claim:

1. A method of preventing or treating disease or disorder comprising administration of oral pharmaceutical composition comprising therapeutically effective amount of PTH analog to a patient in need thereof, wherein said composition provides relative bioavailability of at least 0.5% compare to subcutaneous administration.

2. The method according to claim 1, wherein said oral pharmaceutical composition further comprises at least one degradation preventing agent.

3. The method according to claim 2, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

4. The method according to claim 3, wherein said at least one metal containing compound is selected from group consisting of vanadium sulfate, chromium picolinate and manganese gluconate.

5. The method according to claim 2, wherein said oral pharmaceutical composition comprises PTH analogue and at least one degradation preventing agent in physically separated form.

6. The method according to claim 2, wherein said oral pharmaceutical composition comprises PTH analogue in enteric coated form.

7. The method according to claim 2, wherein said oral pharmaceutical composition comprises PTH analogue selected from teriparatide or abaloparatide.

8. The method according to claim 2, wherein said oral pharmaceutical composition further comprises at least one absorption enhancer.

9. An oral pharmaceutical composition comprising therapeutically effective amount of PTH analog, wherein said composition upon oral administration provides relative bioavailability of at least 0.5% compare to subcutaneous administration.

10. The oral pharmaceutical composition according to claim 9, wherein said composition further comprises at least one degradation preventing agent.

11. The oral pharmaceutical composition according to claim 10, wherein said at least one degradation preventing agent comprises combination of at least one metal containing compound and at least one reducing agent, wherein said at least one metal containing compound is selected from group consisting of vanadium containing compound, chromium containing compound and manganese containing compound, wherein said metal containing compound is in any form including salts or complex thereof.

12. The oral pharmaceutical composition according to claim 11, wherein said at least one metal containing compound is selected from group consisting of vanadium sulfate, chromium picolinate and manganese gluconate.

13. The oral pharmaceutical composition according to claim 10, wherein said composition comprises PTH analogue and at least one degradation preventing agent in physically separated form.

14. The oral pharmaceutical composition according to claim 10, wherein said composition comprises PTH analogue in enteric coated form.

15. The oral pharmaceutical composition according to claim 10, wherein said composition comprises PTH analogue selected from teriparatide or abaloparatide.

16. The oral pharmaceutical composition according to claim 10, wherein said oral pharmaceutical composition further comprises at least one absorption enhancer.
